(19) United States
(12) Patent Application Publication
(10) Pub. No.: US 2025/0281111 A1
(43) Pub. Date: Sep. 11, 2025

(54) SYSTEMS AND METHODS FOR MEASURING THE EFFICACY OF THERAPEUTIC INTERVENTIONS IN DIABETIC SUBJECTS

(71) Applicant: Dr. T.G. Elliott, Inc., Vancouver (CA)

(72) Inventors: Thomas Elliott, Vancouver (CA); Nabeel Khan, Vancouver (CA); Kate Hawke, Vancouver (CA)

(21) Appl. No.: 18/599,482

(22) Filed: Mar. 8, 2024

(51) Int. Cl. A61B 5/00; A61B 5/145; G16H 20/17

(52) U.S. Cl. CPC A61B 5/4848; A61B 5/14532; A61B 5/4839; A61B 5/742; G16H 20/17

(57) ABSTRACT

Described herein are examples of systems and methods for determining the efficacy of a therapeutic intervention related to the treatment of diabetes. In an example, a continuous glucose monitor can be used to monitor the glucose levels of a subject. An insulin pump can administer insulin to the subject in response to the glucose levels obtained by the continuous glucose monitor. The efficacy of a treatment intervention can be determined by comparing an amount of insulin administered to the subject by the insulin pump over a timeframe associated with the treatment intervention to an amount of insulin administered to the subject by the insulin pump over a timeframe associated with a control or placebo period and/or a timeframe associated with a second treatment intervention.

### BACKGROUND

Diabetes is a chronic medical condition that occurs when the body is unable to properly regulate blood sugar (glucose) levels. Glucose is a crucial source of energy for cells, and its levels in the blood are normally controlled by insulin, a hormone produced by the pancreas.

Common symptoms of diabetes include increased thirst, frequent urination, unexplained weight loss, fatigue, blurred vision, and slow wound healing. If left untreated, diabetes can lead to serious complications, such as cardiovascular disease, kidney damage, nerve damage, and vision problems. Management of diabetes involves maintaining blood sugar levels within a target range through a combination of medication, lifestyle changes (including a healthy diet and regular exercise), and monitoring blood glucose levels. Regular medical check-ups and consultations with healthcare professionals are crucial for effective diabetes management.

There are two main types of diabetes: Type 1 diabetes and Type 2 diabetes. Type 1 diabetes is an autoimmune condition where the immune system mistakenly attacks and destroys the insulin-producing cells in the pancreas. As a result, the body is unable to produce enough insulin. People with Type 1 diabetes typically need to take insulin through injections or an insulin pump to manage their blood sugar levels.

In Type 2 diabetes, the body either doesn't produce enough insulin or becomes resistant to its effects. This type of diabetes is more common and is often linked to factors such as obesity, lack of physical activity, and genetic predisposition. Initially, lifestyle modifications, oral medications, and sometimes insulin injections may be used to manage Type 2 diabetes.

Automated Insulin Delivery (“AID”) systems are sometimes used to automatically adjust and deliver insulin to subjects with diabetes. The main components of an AID system typically include: (1) a Continuous Glucose Monitoring (“CGM”) device; (2) an insulin pump; and (3) a control algorithm. The CGM device continuously measures glucose levels in the interstitial fluid under a subject's skin. The CGM sensor can provide real-time data, allowing the system to respond promptly to changes in blood sugar levels. The insulin pump is a device worn externally that typically delivers insulin into the body through a small tube (cannula) placed under the skin. The pump holds a reservoir of rapid-acting insulin, and users (whether the subject or a caregiver) can program it to deliver basal and bolus insulin doses. Basal insulin provides a background level of insulin throughout the day and night, mimicking the continuous release of insulin that occurs in a healthy pancreas. It helps regulate blood sugar levels between meals and during periods of fasting (such as overnight). Bolus insulin, on the other hand, is generally administered with meals to control the rise in blood sugar that occurs after eating. The main contribution of meal-related glucose is from the digestion of carbohydrates. Basal and bolus insulin administered as rapid-acting insulin by an insulin pump can be mimicked with the use of injections of long-acting basal insulin and mealtime bolus insulin in which case it is referred to as basal-bolus therapy.

The control algorithm of an AID system is software that processes information from the CGM device and determines the appropriate amount of insulin (both basal and bolus) to be delivered to a subject. In making such determinations, the algorithm can consider factors such as the subject's current glucose levels, the rate of change in glucose levels (insulin sensitivity), the carbohydrates absorbed through digestion (carbs on board), and previous insulin doses administered to the subject (insulin-on-board).

AID systems aim to mimic the function of a healthy pancreas by adjusting insulin delivery in response to changes in glucose levels. The goal is to keep blood sugar levels within a target range, reducing the risk of hypoglycemia (low blood sugar) and hyperglycemia (high blood sugar).

Many therapeutic interventions have been trialed with the goal of slowing or halting the destruction of pancreatic beta cells (in the case of Type 1 diabetes), avoiding or delaying the onset of clinical Type 1 diabetes, or augmenting the number of beta cells (in Type 1 or Type 2 diabetes) thereby increasing endogenous insulin secretory capacity. Other therapeutic interventions may seek to increase insulin sensitivity, thereby increasing the effectiveness of insulin, or to reduce the requirement of insulin by reducing glucose absorption from the gut or by increasing the excretion of glucose from the kidneys. In practice, all such interventions must be assessed with standardized measures to determine if the intervention is successful in increasing endogenous insulin secretion and/or insulin sensitivity, or reducing exogenous insulin requirements.

In 2004, the American Diabetes Association determined that the appropriate outcome measure for Type 1 diabetes clinical trials to preserve beta-cell function is ‘C-peptide’, a peptide co-secreted with insulin in a one-to-one molar ratio. Laboratory measurement of C-peptide levels under standardized conditions provides a sensitive and clinically-validated assessment of beta-cell function. However, this approach is expensive and logistically challenging, requiring a large number of blood draws from subjects, and there are often delays in determining the effectiveness of an intervention due to laboratory processing times.

As a result, a need exists for improved systems and methods for measuring the effectiveness of therapeutic interventions in the treatment of Type 1 and Type 2 diabetes. In particular, systems and methods are needed that require fewer (if any) blood draws from subjects undergoing therapeutic interventions and/or reduce laboratory processing times.

### SUMMARY

Descriptions herein include examples of systems and methods for evaluating the efficacy of diabetes treatment interventions. In an example, a system can include a CGM device, an insulin pump, and a computing device executing one or more applications. The CGM can be used to monitor the blood glucose levels of a subject. The insulin pump can be configured to responsively administer basal and/or bolus insulin to the subject on an as-needed basis based, in part or primarily, on the subject's blood glucose levels.

In one aspect, during a control phase of an experimental treatment protocol, the amount of insulin administered to the subject by the insulin pump over a duration of time can be monitored and/or recorded. The total amount of insulin administered to the subject by the insulin pump during this control time period can reflect a baseline for the subject's exogenous insulin requirements.

In another aspect, a treatment intervention (e.g., a dose of an insulin, medication, supplement, other substance or intervention the efficacy of which is being evaluated) can be administered to the subject independent of the insulin pump. Similar to the control phase of the protocol, the amount of insulin administered to the subject by the insulin pump over a duration of time associated with administration of the treatment intervention can then be monitored and/or recorded.

In a further aspect, to evaluate the efficacy of the treatment intervention, the total amount of insulin administered to the subject by the insulin pump during the control time period can be compared to the total amount of insulin administered to the subject by the insulin pump during the time period associated with the treatment intervention. Any change in the amount of insulin administered by the insulin pump between the two time periods can serve as a proxy for the effectiveness of the treatment intervention. For example, where the total amount of insulin administered to the subject during the treatment intervention time period is lower than the amount of insulin administered to the subject during the control time period, it can be concluded that the treatment intervention was successful in lowering the subject's exogenous insulin requirements. In this way, the efficacy of the treatment intervention can be evaluated without the need to draw blood from the subject and while greatly reducing laboratory processing times.

In still a further aspect, additional treatment interventions of increasing or decreasing doses of the first intervention and/or alternative insulins, medications, supplements, or other substances can be evaluated. In each case, the amount of insulin administered to the subject by the insulin pump over a duration of time associated with administration of the respective treatment intervention can be monitored, recorded, and then compared to the amount of insulin administered to the subject by the insulin pump during time periods associated with other interventions and/or the control time period.

In further or alternative embodiments, the control phase of the protocol can also be augmented or substituted for a placebo phase in which a placebo is administered to the subject. The amount of insulin administered to the subject by the insulin pump over a duration of time associated with administration of the placebo can then be monitored and/or recorded. This amount of insulin administered to the subject during the placebo phase of the protocol can then be compared to the amount of insulin administered during time periods associated with one or more treatment interventions to assess the efficacy of the interventions.

In another aspect, the one or more applications executed by the computing device can display a graph depicting the amount of insulin administered to the subject by the insulin pump during the control time period, the placebo time period, and/or one or more treatment intervention time periods, all as a function of time. In this way, a user can quickly and intuitively evaluate the efficacy of the one or more trialed treatment interventions at a glance.

Both the foregoing general description and the following detailed description are exemplary and explanatory only and are not restrictive of the examples, as claimed.

### BRIEF DESCRIPTION OF THE DRAWINGS

The accompanying drawings, which are incorporated in and constitute a part of this disclosure, illustrate various embodiments and aspects of the present disclosure. In the drawings:

FIG. 1 is an illustration of example components of an AID system in accordance with this disclosure;

FIG. 2 is an illustration of CGM and insulin pump data collected by an AID system;

FIG. 3 is a flow chart depicting steps in an example process for determining the effectiveness of a therapeutic intervention; and

FIG. 4 is an illustration of data depicting an amount of insulin delivered to a subject during a series of therapeutic interventions.

### DESCRIPTION OF THE EXAMPLES

Reference will now be made in detail to the present examples, including examples illustrated in the accompanying drawings. Wherever possible, the same reference numbers will be used throughout the drawings to refer to the same or like parts.

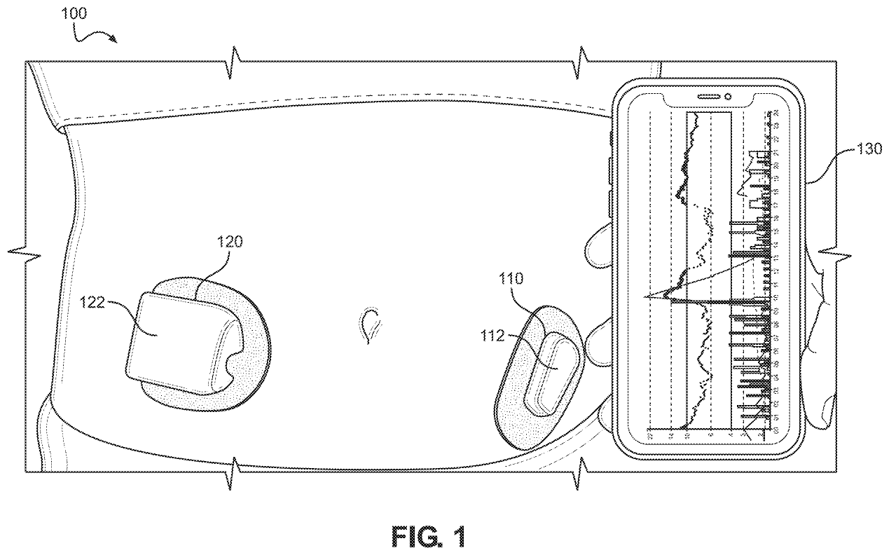

FIG. 1 is an illustration of an AID system 100 for use in assessing the exogenous insulin requirements of a subject and determining the administration of basal and bolus insulin. In one aspect, AID system 100 can include a CGM device 110, an insulin pump 120, and a computing device 130 for executing a treatment application 140. In some examples, the goal of system 100 is to automate the process of insulin delivery, mimicking the function of a healthy pancreas to maintain blood sugar levels within a target range.

In some embodiments, CGM device 110 can include an external housing 112 a subcutaneous sensor 114 (not depicted) for measuring glucose levels in the interstitial fluid of a subject. In use, the CGM device 110 can collect real-time data (e.g., minute-to-minute, hour-to-hour, day-to-day) indicating the current blood sugar level of the subject, as well as rate of change data.

In further embodiments, insulin pump 120 can include a housing 122 that encloses one or more insulin reservoirs 124 (not depicted) containing rapid-acting insulin for administration of basal and bolus insulin. Insulin pump 120 can further include a cannula 124 (i.e., a thin tube, not depicted) that extends from insulin reservoir(s) 124 to a location under the subject's skin. In this way, pump 120 can administer basal and bolus insulin from reservoir(s) 124 to the subject via cannula 124.

In another aspect, AID system 100 includes application 140 executing on computing device 130. Application 140 can process information collected by CGM device 110, including but not limited to current glucose levels in the subject, rate of change data, and/or historical glucose level data. In a further aspect, application 140 can then use this information to make decisions about when and how much basal and/or bolus insulin to deliver to the subject by activating insulin pump 120.

In use, AID system 100 can determine when and how much basal insulin to deliver to a subject to maintain a target blood sugar range between meals and during periods of fasting (e.g., while the subject sleeps). In making its determinations, application 140 can consider trends in the subject's glucose levels and proactively prevent levels that are outside the target range (e.g., too high or too low). Similarly, with respect to bolus insulin, when a subject enters information about meals or snacks (or system 100 predicts the need for bolus insulin based on CGM data), application 140 can calculate the appropriate timing and amount of bolus insulin to administer to the subject to address an anticipated rise in blood sugar associated with food consumption.

In some examples, the AID system 100 is a “closed-loop” system. Such systems can operate substantially autonomously (or semi-autonomously) by making both basal and bolus insulin adjustments without user intervention most of the time. However, while some embodiments of AID system 100 can automate many aspects of insulin delivery, user interaction may still be needed in certain situations. For example, a subject may need to input information to AID system 100 to indicate mealtimes, the performance of exercise, during sickness, and/or make adjustments based on individual preferences and circumstances.

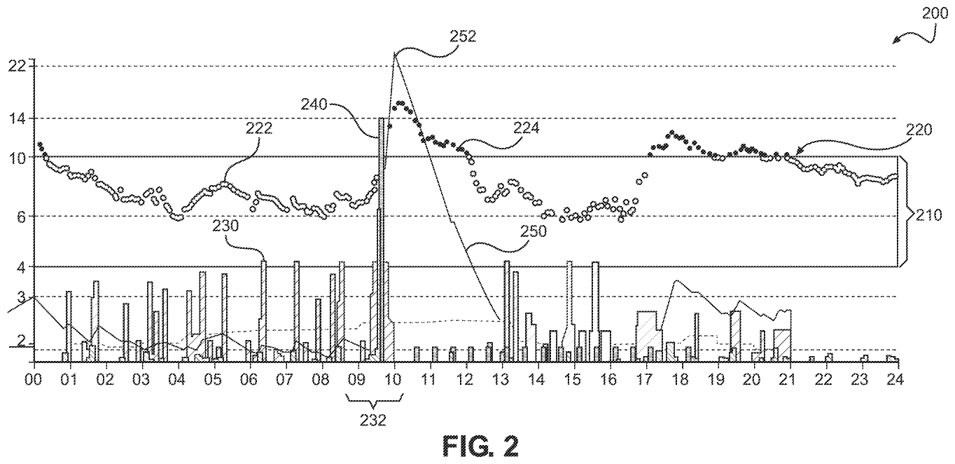

FIG. 2 depicts a graph 200 comprising data collected from the CGM device 110 and the insulin pump 120 as a function of time. In one aspect, graph 200 depicts glucose (e.g., in mmol/L) on the vertical axis and time (e.g., time of day) on the horizontal axis. In some embodiments, graph 200 includes a horizontal band 210 with a lower limit at approximately 4 mmol/L and an upper limit at approximately 10 mmol/L. In some examples, band 210 can represent an acceptable range for a subject's blood glucose levels.

As shown by graph 200, a dotted line 220 can be plotted in which each dot comprising line 220 represents a glucose measurement at a corresponding time, as collected by CGM device 110. As shown, dots located within band 210 (i.e., dots representing a glucose measurement within the acceptable glucose range at the corresponding time) can be shown in a first color or shape 222 (e.g., unfilled dots) and dots located outside band 210 (i.e., dots representing a glucose measurement outside the acceptable glucose range at the corresponding time) can be shown in a second color or shape 224 (e.g., filled dots).

In a further aspect, along the bottom of graph 200, a first series of bars 230 of one color/shading/shape can represent an amount and timing of insulin (basal and/or bolus) administered to the subject through insulin pump 120. In some embodiments, bars 230 representing basal insulin can appear visually distinct (e.g., different color, shading, or shape) from bars 230 representing bolus insulin. A second series of bars 240 of another color/shading/shape can, in some embodiments, represent a carbohydrate entry (e.g., a meal) declared or predicted by AID system 100.

In another aspect, and also along the bottom of graph 200, a line 250 depicts an estimate of the amount of “insulin on board” for the subject, i.e., an amount of insulin in the subject's body as delivered by AID system 100.

As depicted in FIG. 2, it can be determined from bar 240 that the subject consumed a meal (approximately 74 grams of carbohydrates) at about 9:30 am. In one aspect, a spike in measured glucose occurs at or immediately following the meal, as depicted by dotted line 220 rising above the upper limit of band 210. In another aspect, and in response to the detected spike in glucose, a cluster 232 of bars can be observed which are indicative of the administration of insulin to the subject through insulin pump 120. The cluster 232 of bars indicative of the administration of insulin also causes a temporary peak 252 in line 250 indicative of insulin on board the subject.

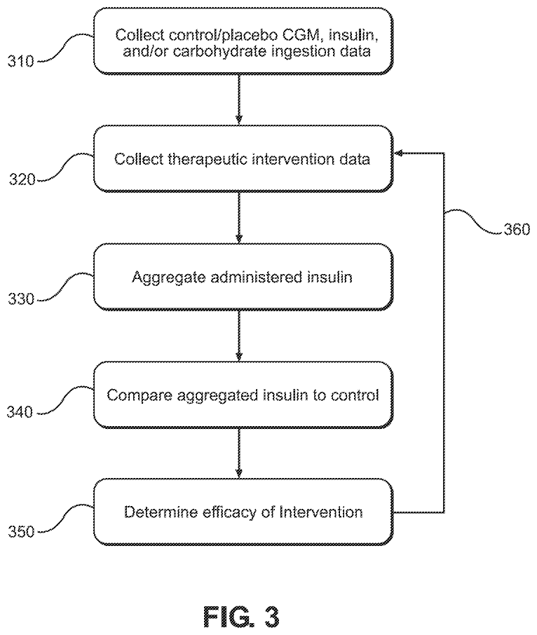

FIG. 3 depicts an illustrative example of a process for determining the efficacy of a therapeutic intervention without the need to draw blood from the subject and while reducing any laboratory processing time. In one aspect, at step 310, control data can be collected from a subject prior to the administration of any treatment interventions. In one embodiment, control data can include the data described with respect to FIG. 2. For example, control data for the subject can include data collected from the subject's CGM device and insulin pump. In further embodiments, control data can include the subject's blood glucose level, insulin administration data (basal and/or bolus), carbohydrate entry (i.e., meal) data, and/or insulin on board data, all with respect to time (e.g., minute-to-minute, hour-to-hour). In one example, control data can be collected on a first day of an experimental protocol. In a further example, control data can be collected in a timeframe that includes the subject's ingestion of a meal.

In a further aspect, step 310 may further comprise collecting placebo data. In some embodiments, a placebo (e.g., saline) can be administered to the subject as part of the experimental protocol and the same type(s) of data can be collected that were collected with respect to the aforementioned control data. In further embodiments, the placebo can be administered to the subject on a second day of the experimental protocol, for example, under substantially similar conditions as those present for the control data (e.g., the placebo data can be collected at approximately the same time of day and/or approximately the same time before/after a meal as the previously described control data). In alternative embodiments, the placebo administration and placebo data collection can be performed as an alternative to collection of the control data (e.g. on a first day of the experimental protocol).

At step 320, first therapeutic intervention data can be collected. For example, a therapeutic intervention can be administered to the subject independent of the insulin pump and under substantially similar conditions as those present during collection of the control and/or placebo data. The therapeutic intervention can be any suitable or experimental intervention for a diabetic or pre-diabetic subject. For example, the therapeutic intervention can be an “Insulin X” administered as a single subcutaneous injection fifteen minutes prior to a test meal. The experimental protocol may further contemplate gradually increasing the amount of Insulin X administered to the subject and determining the efficacy of each dose, as described below. In alternative embodiments, the therapeutic intervention can be some other type of insulin, medication, supplement, or other substance for which the effectiveness in the subject is being evaluated.

In a further aspect, the same type(s) of data can be collected in a timeframe that captures the first therapeutic intervention and for a duration substantially similar to the timeframes over which control and/or placebo data were collected. For example, the first therapeutic intervention can be administered on a third day of the experimental protocol and at approximately the same time of day as the control and/or placebo data collection and/or at approximately the same amount of time before or after a meal.

In another aspect, the subject's AID system continues to monitor the subject's blood glucose levels during the control, placebo, and first therapeutic intervention timeframes. In response to the detected blood glucose levels, the AID system also continues to administer insulin to the subject on an as-needed basis. At step 330, insulin administration data collected from the insulin pump can be aggregated for timeframes associated with one or more of the control data, the placebo data, and the first therapeutic intervention data. For example, in some embodiments, administered insulin can be aggregated for each of the control, placebo, and first therapeutic intervention data over a selected timeframe commencing at a first time of day (e.g., 9:00 am) and/or an amount of time before or after a meal (e.g., fifteen minutes before a meal) and terminating at a second time of day or after a selected duration (e.g., eight hours after commencement or eight hours after the meal). In further embodiments, aggregated administered insulin can be a combination of basal and bolus insulin or the aggregated administered insulin can be separated into the two aggregate amounts for each type of administered insulin. In still further embodiments, aggregated administered insulin can be calculated by, for example, summing the amounts of administered insulin represented by one or more bars 230 depicted in FIG. 2.

In this way, at step 340, aggregated insulin administered to the subject through the subject's AID system during the first therapeutic intervention can be directly compared to the aggregated insulin administered to the subject through the subject's AID system during the control and/or placebo timeframes.

These comparisons can then be used, at step 350, to determine the efficacy of the first therapeutic intervention. For example, where aggregated insulin administered to the subject by the subject's AID system over the timeframe associated with the first therapeutic intervention is lower than the aggregated insulin administered to the subject by the AID system over the timeframes associated with the control data and/or the placebo data, it can be concluded that the first therapeutic intervention was successful (i.e., reduced the exogenous insulin requirements of the subject when compared to the absence of the therapeutic intervention).

In a further aspect, at step 360, subsequent or iterative therapeutic interventions can be assessed on, for example, subsequent days and under substantially similar conditions, to find an ideal dose and/or insulin treatment for the subject. For example, second, third, and fourth therapeutic interventions of Insulin X can be administered to the subject on subsequent days and in increasing doses. In some embodiments, the total or aggregated amount of insulin administered to the subject during the relevant timeframes of the subsequent interventions can be compared to prior and/or future interventions to determine an ideal dosage of Insulin X for the subject and/or identify a point of diminishing returns with respect to the dosage of Insulin X for the subject.

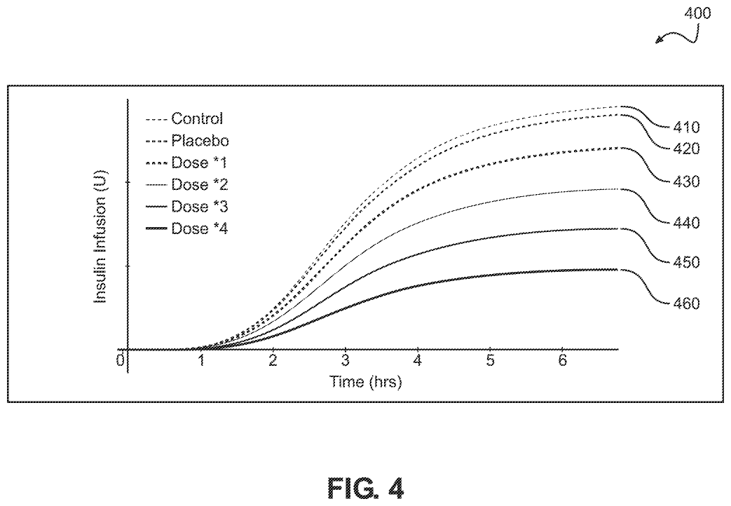

FIG. 4 depicts an example embodiment of a graph 400 for providing comparison data for one or more therapeutic interventions, as well as control and/or placebo data, for a subject. In one aspect, graph 400 depicts aggregated administered insulin (in U) administered to the subject by the subject's AID system on the vertical axis and time (e.g., in hours) on the horizontal axis. In another aspect, graph 400 depicts lines 410 and 420 representing aggregated administered insulin as a function of time for each of control data and placebo data. Data associated with one or more therapeutic interventions are also shown on graph 400. For example, in the embodiment depicted in FIG. 4, aggregated insulin amounts administered to the subject in timeframes associated with four serial therapeutic interventions (e.g., doses 1, 2, 3, and 4) are represented as lines 430, 440, 450, and 460. Each intervention can be, for example, associated with an increased dose of Insulin X relative to the prior intervention.

In a further aspect, a comparison of lines 430, 440, 450, and 460 to one another and control line 410 and placebo line 420 can be used to determine the efficacy of the four therapeutic interventions. For example, in the embodiment depicted in FIG. 4, all interventions represent an improvement (i.e., reduced the exogenous insulin requirements of the subject) compared to the subject's control and/or placebo data. Moreover, each subsequent intervention (representing in this case an increased dose of Insulin X) represents an improvement compared to prior interventions in terms of total aggregate insulin administered to the subject. In a further aspect, and as described previously, while FIG. 4 does not break down the aggregate administered insulin for each relevant timeframe into basal and bolus insulin, one of ordinary skill would appreciate that FIG. 4 could be modified to present such data and such a breakdown in the types of administered insulin are within the scope of this disclosure.

Other examples of the disclosure will be apparent to those skilled in the art from consideration of the specification and practice of the examples disclosed herein. Though some of the described methods have been presented as a series of steps, it should be appreciated that one or more steps can occur simultaneously, in an overlapping fashion, in a different order, or a different timeframe. The order of steps presented is only illustrative of the possibilities and those steps can be executed or performed in any suitable fashion. Moreover, the various features of the examples described here are not mutually exclusive. Rather, any feature of any example described here can be incorporated into any other suitable example. It is intended that the specification and examples be considered as illustrative only, with a true scope and spirit of the disclosure being indicated by the following claims.

What is claimed is:

1. A system for determining the efficacy of a therapeutic intervention related to diabetes, comprising:
a continuous glucose monitoring device for measuring a subject's blood glucose levels;
an insulin pump for administering insulin to the subject in response to one or more blood glucose measurements received from the continuous glucose monitoring device; and
a computer-readable medium containing instructions that, when executed by a hardware-based processor, performs stages, including:
receiving, from the insulin pump, a first plurality of insulin measurements indicative of insulin administered to the subject during a control phase of an experimental protocol;
receiving a second plurality of insulin measurements indicative of insulin administered to the subject during an intervention phase of the experimental protocol;
aggregating the first plurality of insulin measurements to determine a first amount of insulin administered to the subject during the control phase;
aggregating the second plurality of insulin measurements to determine a second amount of insulin administered to the subject during the intervention phase; and
comparing the first amount of insulin to the second amount of insulin to determine whether the intervention was successful.

2. The system of claim 1, wherein the receiving the first plurality of insulin measurements includes receiving a first timeframe corresponding to at least a portion of the control phase of the experimental protocol.

3. The system of claim 2, wherein the receiving the second plurality of insulin measurements includes receiving a second timeframe corresponding to at least a portion of the intervention phase of the experimental protocol.

4. The system of claim 3, wherein the intervention was successful if the second amount of insulin is less than the first amount of insulin.

5. The system of claim 4, wherein the first timeframe is associated with a first day of the experimental protocol the second timeframe is associated with a second day of the experimental protocol.

6. The system of claim 1, the stages further comprising displaying a graph depicting the aggregated first and second amounts of administered insulin as a function of time.

7. The system of claim 1, wherein the first and second plurality of insulin measurements each include one or more basal insulin measurements and one or more bolus insulin measurements.

8. A method for assessing the efficacy of a therapeutic intervention related to diabetes, the method including:
receiving, from an insulin pump associated with a subject, a first plurality of insulin measurements indicative of insulin administered to the subject during a control timeframe of a test protocol;
receiving, from the insulin pump, a second plurality of insulin measurements indicative of insulin administered to the subject during an intervention timeframe of the test protocol;
aggregating the first plurality of insulin measurements to determine a first amount of insulin administered to the subject during the control timeframe;
aggregating the second plurality of insulin measurements to determine a second amount of insulin administered to the subject during the intervention timeframe; and
comparing the first amount of insulin to the second amount of insulin to determine whether the subject's exogenous insulin requirements were lower during the intervention timeframe than during the control timeframe.

9. The method of claim 8, wherein a duration of the control timeframe is approximately the same as a duration of the intervention timeframe.

10. The method of claim 9, wherein the control timeframe takes place on a first day and the intervention timeframe takes place on a second day.

11. The method of claim 8, further comprising displaying a chart depicting the aggregated first and second amounts of administered insulin as a function of time.

12. The method of claim 8, wherein the intervention reduced the subject's exogenous insulin requirements if the second amount of insulin is less than the first amount of insulin.

13. The method of claim 8, further comprising:
receiving, from the insulin pump, a third plurality of insulin measurements indicative of insulin administered to the subject during a second intervention timeframe of the test protocol;
aggregating the third plurality of insulin measurements to determine a third amount of insulin administered to the subject during the second intervention timeframe; and
comparing the third amount of insulin to the first or second amounts of insulin to determine whether the subject's exogenous insulin requirements were lower during the second intervention timeframe than during the first intervention timeframe or the control timeframe.

14. The method of claim 8, wherein the first and second plurality of insulin measurements each include one or more basal insulin measurements and one or more bolus insulin measurements.

15. A method for assessing the efficacy of a therapeutic intervention administered during a diabetes treatment protocol, the method including:
receiving, from an insulin pump, a first amount of insulin administered to a subject by the insulin pump during a first timeframe associated with an administration of a placebo to a subject;
receiving, from the insulin pump, a second amount of insulin administered to the subject by the insulin pump during a second timeframe associated with an administration of a first dose of an intervention to the subject;
comparing the first amount of insulin to the second amount of insulin to determine whether the subject's exogenous insulin requirements were lower during the second timeframe than the first timeframe.

16. The method of claim 15, further comprising:
receiving, from the insulin pump, a third amount of insulin administered to the subject by the insulin pump during a third timeframe associated with an administration of a second dose of the intervention to the subject;
comparing the third amount of insulin to the first or second amounts of insulin to determine whether the subject's exogenous insulin requirements were lower during the third timeframe than during the first or second timeframes.

17. The method of claim 16, wherein the second dose of the intervention is greater than the first dose of the intervention.

18. The method of claim 15, wherein the first and second amounts of insulin each include an amount of basal insulin and an amount of bolus insulin.

19. The method of claim 15, wherein the first and second timeframes take place on different days.

20. The method of claim 15, further comprising displaying a chart depicting the first and second amounts of insulin as a function of time.
